(19) United States
(12) Patent Application Publication
(10) Pub. No.: US 2014/0276061 A1
(43) Pub. Date: Sep. 18, 2014

(54) System and Method for Identification of Breast Ductal Network

(71) Applicant: Cubuus, Inc., Redwood City, CA (US)

(72) Inventors: Roberta Lee, Redwood City, CA (US); Kio Kim, Daly City, CA (US)

(73) Assignee: Cubuus, Inc., Redwood City, CA (US)

(21) Appl. No.: 14/216,898

(22) Filed: Mar. 17, 2014

Related U.S. Application Data
(60) Provisional application No. 61/799,201, filed on Mar. 15, 2013.

(51) Int. Cl. A61B 10/00; A61B 5/00; A61B 8/08

(52) U.S. Cl. CPC A61B 10/0041; A61B 8/0825; A61B 5/0091; A61B 5/0051; A61B 5/742
USPC 600/443; 600/407; 600/476; 600/587; 600/552

(57) ABSTRACT

An imaging system induces an increased visibility of the fluid-containing ducts of the breast to enhance the imaging of the ducts, to improve differentiation of the ducts from the surrounding tissue and to detect lesions present within the duct. The imaging system includes a noninvasive fluid visibility increasing module configured for increasing the visibility of a fluid contained within one or more ducts of the breast and an increased fluid visibility detector configured for detecting the one or more ducts having increased fluid visibility to identify the one or more ducts within the ductal system of the breast. The imaging system further includes an image processor capable of rendering a three-dimensional map of the ductal system of the breast.

### CROSS REFERENCE TO RELATED APPLICATIONS

This application claims priority to U.S. Provisional Patent Application No. 61/799,201, titled “Imaging of Mammary Gland for Detection and Guidance of Interventional Procedures”, filed Mar. 15, 2013, incorporated herein by reference in its entirety.

### BACKGROUND OF THE INVENTION

Various techniques are known in the art for the assessment of breast tissue and the detection of breast cancer. These techniques include mammography, magnetic resonance imaging (MRI) and conventional ultrasound imaging. Mammography, which is an x-ray of the breast, is typically focused on early detection of breast cancer and relies on identification of abnormal shapes, distortions and/or microcalcifications. Microcalcifications are tiny specks of mineral deposits, such as calcium, that may be scattered throughout the breast tissue. However, mammography, MRI and conventional ultrasound techniques are not capable of identifying the underlying anatomy of the ducts and ductal system within the breast tissue. Moreover, mammography and MRI are not applicable to real-time imaging guidance for interventional procedures. Identification of the ducts and the rendering of a three-dimensional map of the duct network of the breast would greatly improve detection, localization and excision of abnormal lesions within the ductal system.

The human breast consists of 15 to 20 lobes that begin centrally beneath the nipple-areolar complex and extend to the periphery of the gland. Milk is produced in numerous small terminal ductal lobular units (TDLUs) and flows through small ducts which drain into one or more main ducts that open on the surface of the nipple. Breast cancer originates from stem cells within or near the TDLU. Breast cancer often spreads within the lumen of the ducts of the involved lobe, before or in addition to, spreading out of the duct system and into the surrounding breast tissue as invasive breast cancer. The spread of cancer within the lumen of the duct, known as ductal carcinoma in situ (DCIS), is not detectable by mammographic imaging unless microcalcifications are present within the duct. Conventional ultrasound may detect microcalcifications within the breast tissue by using high frequency imaging, however the ducts themselves or lesions within the duct are often not identified using conventional high frequency ultrasound because this requires manual alignment of the ultrasound transducer with the underlying duct. DCIS lesions are sometimes detected using MRI imaging, but as previously indicated, neither MRI nor mammography provides real-time imaging guidance during interventional procedures. These limitations of current imaging modalities lead to delayed detection and diagnosis of breast cancer and limit the accuracy of interventional procedures.

An ultrasound examination of the milk ducts within the breast as described in U.S. Pat. No. 5,709,206 to Teboul, incorporated herein by reference, can be utilized to study the lumens of milk ducts and the anatomical pattern of the ducts in relation to the lobe in which they are contained. In accordance with Teboul, by using axial ductal ultrasound imaging, ducts within the breast tissue can be identified. However, the manual technique of axial ductal ultrasound imaging is difficult to learn and viewed images are processed internally by the operator to obtain a mental three-dimensional rendition of the duct anatomy.

Another ultrasound examination of the milk ducts within breast tissue is described in U.S. Pat. No. 6,736,781 to Lee, incorporated herein by reference. In accordance with Lee, an ultrasound contrast agent is injected into the milk duct to enhance ultrasound imaging of the duct. However, dilating the opening of the milk duct and injecting the contrast agent into the duct is technically difficult, invasive and often painful for the patient.

A further ultrasound examination of the ducts within the breast tissue is described in U.S. Pat. No. 8,376,947 to Rambod et al., incorporated herein by reference. In accordance with Rambod, a modulated ultrasound frequency is applied to breast tissue to stimulate a differentiation in response frequency. Soft breast tissue emits a response frequency of 1-50 Hz and microcalcifications emit frequencies in a range of 100 Hz to 100 kHz when subjected to a modulated ultrasound frequency. Lesions within the breast tissue, however, and in particular within the ducts of the breast, do not necessarily contain microcalcifications and would potentially be missed using this method.

Acoustic radiation force imaging has been described in the literature to image lesions within the breast tissue such as cysts, cancer and fibroadenomas. The acoustic radiation force imaging known in the art has been limited to the characterization of isolated lesions within the breast tissue without regard to the relation of the lesions to the anatomical structure of the milk ducts and the associated ductolobular system of the breast tissue.

Ductolobular systems and small lesions within the ducts may be difficult to identify in part due to limitations of ultrasound imaging. Conventional ultrasound imaging relies on the skill of the operator who may not be adept at imaging or identifying milk ducts within the breast tissue. Additionally, if the ultrasound transducer is not aligned with the length of a duct, the duct will be imaged in cross-section and will be difficult to identify.

Gooding et al (J Ultrasound Med. 2010 January; 29(1):95-103) delineated sections of the intraductal network of breast tissue by performing ultrasound imaging of lactating women. Breast ducts are easier to visualize by ultrasound in lactating women as the ducts are dilated with milk. However, this technique is not feasible in the breasts of non-lactating women as the nondilated ducts would be significantly more difficult to identify.

Accordingly, there is a need for an improved system and method for imaging and visualization of the milk ducts within the breast tissue. Preferably, the system utilizes ultrasound imaging of breast tissue to create an individualized map of the milk duct network of the specific breast being imaged. The system and method should improve characterization of a lesion or lesions within the duct system of the breast and improve real-time guidance of interventional procedures. The improved system and method of ultrasound imaging will ideally facilitate accuracy in imaging the duct system of the breast and for improved detection, diagnosis and interventional image-guided procedures of a lesion or lesions within one or more ducts of the ductolobular system of the breast.

### SUMMARY

A system and method for imaging of breast tissue by increasing the visibility of a fluid-containing milk duct within a region in the breast tissue to enhance the ability to distinguish the fluid-containing milk ducts from the surrounding soft tissue. The creation of a three-dimensional map of the ductal system from captured images of the plurality of ducts and the ability to relate the one or more milk ducts to the anatomy of the soft tissue is provided. The ability to detect or improve characterization of a lesion or lesions within the fluid-containing ducts as well as ultrasound imaging guidance of interventional procedures are disclosed.

In one embodiment, a system for identifying one or more ducts within a ductal system of a breast includes a noninvasive fluid visibility increasing module configured to increase the visibility of the fluid contained within one or more ducts of the breast and an increased fluid visibility detector configured to detect the fluid and to identify the ducts with the ductal system of the breast. The system may further include an image processor for capturing a plurality of images provided by the increased fluid visibility detector and a monitor to display the plurality of images to a user of the system.

In a specific embodiment, an ultrasound imaging system uses acoustic waves to enhance the imaging of breast tissue to improve identification and delineation of milk ducts in relation to the anatomy of the milk duct network as a whole and detection of a possible lesion or lesions within the duct. A system for identifying one or more ducts within a ductal system of a breast, the system includes, a vibration inducer configured for inducing vibration of a fluid contained within one or more ducts of the breast and a vibration detector configured for detecting the vibration of the fluid within the one or more ducts to identify the one or more ducts within the ductal system of the breast.

In a specific embodiment, a method for identifying one or more ducts within a ductal system of a breast includes, inducing vibration of a fluid contained within one or more ducts of the breast and detecting the vibration of the fluid within the one or more ducts to identify the one or more ducts within the ductal system of the breast.

In an additional embodiment, a method for identifying one or more ducts within a ductal system of a breast includes, increasing the volume of the fluid contained within one or more ducts of the breast and detecting the increased fluid volume within the one or more ducts to identify the one or more ducts within the ductal system of the breast. The fluid volume may be increased utilizing a fluid volume increasing chemical, drug or hormone, such as oxytocin, or alternatively, the fluid volume may be increased by applying heat to the breast. Alternatively, the fluid volume may be increased utilizing a duct dilating chemical, drug or hormone.

In a further embodiment, the noninvasive fluid visibility increasing module may be comprised of a combination of one or more of the acoustic waves, vibration, increasing fluid volume and duct dilation embodiments.

The present invention provides a system and method for the identification of the breast ductal network utilizing a noninvasive fluid visibility increasing module and an increased fluid visibility detector. The ability to identify the ductal network facilitates improved accuracy in detection, diagnosis and image-guided interventional procedures of a lesion or lesions within the duct system of the breast or similar fluid-containing structures in the human body. Additionally, the ability to identify the ducts within the breast tissue provides for the rendering of a three-dimensional map of the ductal network.

### BRIEF DESCRIPTION OF THE DRAWINGS

In order that the present invention may be more clearly ascertained, some embodiments will now be described, by way of example, with reference to the accompanying drawings, in which:

FIG. 1 is a block diagram of the system in accordance with an embodiment of the present invention.

FIG. 2 is a diagrammatic view illustrating a system for identifying a ductal network within the breast in accordance with an embodiment of the present invention.

FIG. 3 illustrates a diagrammatic view of an exemplary embodiment of the ultrasound transducer device in accordance with an embodiment of the present invention.

FIG. 4 is a flow diagram illustrating a method for identifying a ductal network with the breast in accordance with an embodiment of the present invention.

### DETAILED DESCRIPTION OF THE INVENTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

With reference to FIG. 1, a system 100 for identifying one or more ducts within a ductal system of a breast is provided. The system 100 includes a noninvasive fluid visibility increasing module 105 configured for increasing the visibility of a fluid contained within one or more ducts of the breast and an increased fluid visibility detector 110 configured for detecting the one or more ducts having increased fluid visibility and identifying the one or more ducts within the ductal system of the breast. The system may further include an image processor 115 coupled to the increased fluid visibility detector 110. The image processor 115 is configured to capture images provided by the increased fluid visibility detector 110 and a monitor 120 coupled to the image processor 115 is configured for displaying the captured images to a user of the system 100. The monitor 120 may be configured as a free-standing unit, a visor or a transparent shield that is placed over the one or more ducts of the breast.

In one embodiment, the noninvasive fluid visibility increasing module 105 and the increased fluid visibility detector 110 may be incorporated into a common transducer device 125. In an alternate embodiment, the noninvasive fluid visibility increasing module 105 and the increased fluid visibility detector 110 may be separate devices. The transducer device 125 may be physically coupled to the image processor 115. Alternatively, the transducer 125 may be coupled wirelessly to the image processor 115.

In a particular embodiment, the noninvasive fluid visibility increasing module 105 is a vibration inducer configured for inducing vibration of the fluid contained within one or more ducts of the breast. In this embodiment, the increased fluid visibility detector 110 is a vibration detector for detecting the vibration induced by the vibration inducer. As shown with reference to FIG. 2, a system 200 for identifying one or more ducts with a ductal system of a breast includes a vibration inducer to induce vibration of a fluid 224 within one or more ducts 236a, 236b, 236c that are present within the breast tissue of the breast 226. In general, the milk ducts 236a, 236b, 236c contain a fluid 224 and the ducts are contained within a lobular system and surrounded by soft tissue 220. In this particular embodiment, the vibration inducer and the vibration detector are incorporated into a common transducer device 210 as previously described. In operation, the transducer device 210 is placed in contact with the surface of the breast 226. The vibration inducer of the transducer device 210 is operated to induce a vibration within the fluid 224 contained within one or more ducts 236a, 236b, 236c.

In a particular embodiment, the noninvasive fluid visibility increasing module 105 may be an acoustic signal inducer that is placed in contact with the surface of the breast 226. The acoustic signal inducer is operated to induce an acoustic signal or force within the breast tissue 220 and fluid-containing ducts 236a, 236b, 236c. The induced acoustic signal causes the fluid 224 within the ducts 236a, 236b, 236c to vibrate. While the acoustic signal may also cause vibration in the surrounding soft tissue 220, due to the structural differences between the soft tissue 220 and the fluid 224 within the ducts 236a, 236b, 236c, the vibration of the fluid 224 can be differentiated from the vibration of the soft tissue 220. In general, the vibration inducer will induce less vibration in the soft tissue 220, relative to the vibration induced within the fluid 224 of the ducts 236a, 236b, 236c. The vibrations induced within the fluid 224 may be of various forms and/or frequencies, including but not limited to, pulsatile and turbulent vibrations. In a particular embodiment, the acoustic signal inducer may generate a low-frequency acoustic signal, which may be in the range of less than 1 kHz to about 5 MHz. The emitted low-frequency acoustic signal creates differential movement between the fluid 224 contained within the ducts 236a, 236b, 236c and the surrounding tissue. The acoustic waves may be generated by one or more frequencies in unison, alternating or any other pattern, or alternatively may be randomly generated.

In an additional embodiment, the noninvasive fluid visibility increasing module 105 may be a physical force inducer. In an exemplary embodiment, the physical force inducer may be configured for applying a gentle pulsating force to the breast to induce a vibration within the fluid 224 of the ducts 236a, 236b, 236c.

In a particular embodiment, the increased fluid visibility detector 110 is an ultrasound imaging detector. In operation, the ultrasound imaging detector captures images of the ducts within the breast as the user guides the transducer device 210 over the breast 226 in a predetermined pattern. In an alternate embodiment, the guidance of the transducer device 210 may be performed by mechanical means or may be performed free-hand, with or without following a predetermined pattern.

In additional embodiments, the increased fluid visibility detector 105 may be an optical imaging detector, a motion sensing detector or a Doppler ultrasound imaging detector. In general, the vibration detector is configured to detect the vibration of the fluid 224 within the ducts 236a, 236b, 236c that has been induced by the vibration inducer. The differential movement or vibration of the fluid 224 may be detected visually by an operator of the imaging system or alternatively, may be detected utilizing specialized software designed to detect vibration or differential movement.

The transducer device 210 is coupled to an image processor 115 as previously described with reference to FIG. 1. The image processor 115 is configured to capture a plurality of images provided by the transducer device 210 and to display the images on a monitor 120 to be viewed by a user of the system 200. In an additional embodiment, the image processor 115 is further configured to generate a three-dimensional map of the ductal system of the breast from the plurality of captured images. The image processor 115 may include processing software for rendering a three-dimensional image from the two-dimensional images of the fluid-containing ducts captured by the vibration detector of the transducer device 210. The three-dimensional image may delineate a portion of the ductal system of the breast or the entire ductal system of the breast.

In an additional embodiment, the vibration detector is further configured for detecting a variation in the vibration of the fluid 224 within the one or more ducts to identify the presence of a lesion within the one or more ducts. The presence of a lesion within a duct 236a, 236b, 236c may cause a variation in the vibration of the fluid 224 of the duct that is detectable by the vibration detector. As such, the system of the present invention is capable of assisting in the detection of cancerous lesions within the ducts 236a, 236b, 236c of the breast 226.

To increase the visibility of the fluid-containing ducts 236a, 236b, 236c the noninvasive fluid visibility increasing module 105 may include a fluid volume increasing compound to be administered to the breast 226 prior to detecting the increased fluid visibility of a fluid contained within one or more ducts 236a, 236b, 236c of the breast 226. The fluid volume increasing compound may be one of a chemical, drug or hormone. In a particular embodiment, the fluid volume increasing hormone is oxytocin, commonly known as a milk let-down hormone, which is administered to the breast 226 in one or more of several alternative methods such as orally, intraveneously or topically, prior to the detection of the ducts and imaging of the breast 226. The effects of the oxytocin may increase the fluid volume with the ducts 236a, 236b, 236c, thereby enhancing imaging of the ducts 236a, 236b, 236c. Alternatively, a duct dilating compound may be administered to dilate the one or more ducts to increase visibility of the fluid-containing ducts. The duct dilating compound may be a chemical, drug or hormone. Administration of the duct dilating compound may be performed orally, intravenously or topically.

In another embodiment to increase the visibility of the fluid-containing ducts 236a, 236b, 236c the noninvasive fluid visibility increasing module 105 may include a heat source configured to administer heat to the breast 226 prior to and/or during detecting the fluid within the one or more ducts 236a, 236b, 236c of the breast 226. The heat may be administered to a specific area of interest in the breast 226 or may be administered generally to the entire breast 226. The heat may be administered external or internal to the breast 226.

In a further embodiment to increase the visibility of the fluid-containing ducts 236a, 236b, 236c the noninvasive fluid visibility increasing module 105 may comprise a combination of one or more of the acoustic waves, vibration, physical force, fluid-increasing compound and duct dilating compound embodiments.

With reference to FIG. 3, in a particular embodiment in which the noninvasive fluid visibility increasing module 105 is a vibration inducer, the system of the present invention 300 may include a transducer device 305, such as an ultrasound transducer, comprising at least two arrays of elements. A first array of ultrasound elements may partially form the increased fluid visibility detector 110 embodied as a vibration detector and a second array of acoustic wave emitting elements may partially form the noninvasive fluid visibility increasing module 105 embodied as a vibration inducer. The array of ultrasound elements 310a, 310b, . . . 310n emits and receives acoustic waves in a frequency suitable for ultrasound imaging of the breast tissue, such as within a range of 8 MHz to 15 MHz. The array of acoustic elements 315a, 315b, . . . 315n emits acoustic waves at a preferably lower frequency range, such as within a range of approximately less than 1 kHz to 20 kHz. These ranges are intended to be exemplary and not intended to be limiting. Acoustic waves emitted from the acoustic elements 315a, 315b, . . . 315n can selectively create the acoustic waves that cause the vibration within the fluid 224 of the breast 226. The vibration is detected and imaged by the emitting and receiving ultrasound elements 310a, 310b, . . . 310n. The emitting and receiving ultrasound elements 310a, 310b, . . . 310n and the emitting acoustic elements 315a, 315b, . . . 315n may be linearly arranged, as shown in FIG. 3, or may be arranged in various other configurations. The transducer device 305 may be coupled to the image processor 115 through a connecting cable element 320.

With reference to FIG. 4, a method 400 for identifying one or more ducts within a ductal system of a breast includes, noninvasively increasing the visibility of a fluid contained within one or more ducts of the breast 405. In a particular embodiment, a noninvasive fluid visibility increasing module 105 may be used to increase the visibility of the fluid contained within the one or more ducts.

After the visibility of the fluid within the ducts has been increased, the method 400 further includes, detecting the one or more ducts having increased fluid visibility to identify the one or more ducts within the ductal system of the breast 410. In a particular embodiment, an increased fluid visibility detector 110 may be used to detect the one or more ducts having increased fluid visibility to identify the one or more ducts within the ductal system of the breast.

After the ducts have been detected, the increased fluid visibility detector 110 may be used to capture a plurality of images of the identified one or more ducts 415. The plurality of images may then be provided to an image processor 115 for display on a monitor 120. Alternatively, the plurality of images may be provided to the image processor 115 and specialized software designed to detect vibration or differential movement identifies the fluid-containing ducts 236a, 236b, 236c. A three-dimensional map of the ductal system of the breast may be generated from the plurality of captured images 420.

The present invention provides a system and method for the identification of the breast ductal network utilizing a noninvasive fluid visibility increasing module and an increased fluid visibility detector. The ability to identify the ductal network facilitates improved accuracy in detection, diagnosis and image-guided interventional procedures of a lesion or lesions within the duct system of the breast or similar fluid-containing structures in the human body. Additionally, the ability to identify the ducts within the breast tissue provides for the rendering of a three-dimensional map of the ductal network.

While this invention has been described in terms of several embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for identifying one or more ducts within a ductal system of a breast, the method comprising:
noninvasively increasing the visibility of a fluid contained within one or more ducts of the breast; and
detecting the one or more ducts having increased fluid visibility to identify the one or more ducts within the ductal system of the breast.

2. The method of claim 1, wherein noninvasively increasing the visibility of a fluid contained within one or more ducts of the breast further comprises, inducing vibration of a fluid contained within one or more ducts of the breast.

3. The method of claim 2, wherein inducing vibration of a fluid contained within one or more ducts of the breast further comprises, applying an acoustic signal to the breast to induce the vibration of the fluid within the one or more ducts of the ductal system of the breast.

4. The method of claim 3, wherein the acoustic signal is a low-frequency acoustic signal.

5. The method of claim 2, wherein inducing vibration of a fluid contained within one or more ducts of the breast further comprises, applying a physical force to the breast to induce the vibration of the fluid within the one or more ducts of the ductal system of the breast.

6. The method of claim 1, wherein noninvasively increasing the visibility of a fluid contained within one or more ducts of the breast further comprises administering a fluid volume increasing compound to increase the volume of fluid within the ducts of the breast.

7. The method of claim 6, wherein the fluid volume increasing compound includes at least one of a chemical, a drug and a hormone.

8. The method of claim 7, wherein the fluid volume increasing compound is administered topically, orally or intravenously.

9. The method of claim 1, wherein noninvasively increasing the visibility of a fluid contained within one or more ducts of the breast further comprises administering a duct dilating compound.

10. The method of claim 1, wherein noninvasively increasing the visibility of a fluid contained within one or more ducts of the breast further comprises administering heat to the breast to increase the volume of fluid within the ducts of the breast.

11. The method of claim 1, wherein noninvasively increasing the visibility of a fluid contained within one or more ducts of the breast comprises a combination of one or more of applying acoustic waves, vibration, physical force, a fluid-increasing compound and a duct dilating compound.

12. The method of claim 1, wherein detecting the one or more ducts having increased fluid visibility to identify the one or more ducts within the ductal system of the breast further comprises, performing ultrasound imaging of the breast to detect the one or more ducts having increased fluid visibility to identify the one or more ducts within the ductal system of the breast.

13. The method of claim 1, wherein detecting the one or more ducts having increased fluid visibility to identify the one or more ducts within the ductal system of the breast further comprises, performing optical imaging of the breast to detect the one or more ducts having increased fluid visibility to identify the one or more ducts within the ductal system of the breast.

14. The method of claim 1, wherein detecting the one or more ducts having increased fluid visibility to identify the one or more ducts within the ductal system of the breast further comprises, performing motion sensing of the breast to detect the one or more ducts having increased fluid visibility to identify the one or more ducts within the ductal system of the breast.

15. The method of claim 1, wherein detecting the one or more ducts having increased fluid visibility to identify the one or more ducts within the ductal system of the breast further comprises, performing Doppler ultrasound imaging of the breast to detect the one or more ducts having increased fluid visibility to identify the one or more ducts within the ductal system of the breast.

16. The method of claim 1, wherein detecting the one or more ducts having increased fluid visibility to identify the one or more ducts within the ductal system of the breast further comprises, utilizing specialized software configured to differentiate movement in the fluid of the ducts to detect the one or more ducts having increased fluid visibility to identify the one or more ducts within the ductal system of the breast.

17. The method of claim 1, further comprising:
capturing a plurality of images of the identified one or more ducts; and
generating a three-dimensional map of the ductal system of the breast from the plurality of captured images.

18. The method of claim 1, further comprising, detecting a variation in the fluid within the one or more ducts to identifying the presence of a lesion within the one or more ducts.

19. A system for identifying one or more ducts within a ductal system of a breast, the system comprising:
a noninvasive fluid visibility increasing module configured for increasing the visibility of a fluid contained within one or more ducts of the breast; and
an increased fluid visibility detector configured for detecting the one or more ducts having increased fluid visibility to identify the one or more ducts within the ductal system of the breast.

20. The system of claim 19, wherein the noninvasive fluid visibility increasing module is a vibration inducer configured for inducing vibration of a fluid contained within one or more ducts of the breast

21. The system of claim 20, wherein the vibration inducer is an acoustic signal inducer.

22. The system of claim 21, wherein the acoustic signal inducer is a low-frequency acoustic signal inducer.

23. The system of claim 20, wherein the vibration inducer is a physical force inducer.

24. The system of claim 19, wherein the noninvasive fluid visibility increasing module comprises a fluid volume increasing compound to be administered topically, orally or intravenously to increase the volume of fluid within the ducts of the breast.

25. The system of claim 19, wherein the noninvasive fluid visibility increasing module comprises a duct dilating compound to be administered topically, orally or intravenously to increase the volume of fluid within the ducts of the breast.

26. The system of claim 19, wherein the noninvasive fluid visibility increasing module includes at least one of an acoustic signal inducer, a low-frequency acoustic signal inducer, a vibration inducer, a physical force inducer, a fluid volume increasing compound and a duct dilating compound.

27. The system of claim 19, wherein the noninvasive fluid visibility increasing module comprises a heat source configured to administer heat to the breast to increase the volume of fluid within the ducts of the breast.

28. The system of claim 19, wherein the increased fluid visibility detector is a vibration detector.

29. The system of claim 19, wherein the increased fluid visibility detector is an ultrasound imaging detector.

30. The system of claim 19, wherein the increased fluid visibility detector is an optical imaging detector.

31. The system of claim 19, wherein the increased fluid visibility detector is a motion sensing detector.

32. The system of claim 19, wherein the increased fluid visibility detector is a Doppler ultrasound imaging detector.

33. The system of claim 19, wherein the increased fluid visibility detector comprises software configured to differentiate movement of the fluid within the ducts.

34. The system of claim 19, further comprising an image processor coupled to the increased fluid visibility detector, the image processor configured to capture a plurality of images of the identified one or more ducts.

35. The system of claim 34, further comprising a monitor coupled to the image processor, the monitor to display the plurality of images captured by the image processor.

36. The system of claim 35, wherein the monitor includes at least one of a free-standing unit, a visor and a transparent shield configured to overlay the area being imaged.

37. The system of claim 34, wherein the image processor is further configured to generate a three-dimensional map of the ductal system of the breast from the plurality of captured images.

38. The system of claim 19, wherein the increased fluid visibility detector is further configured for detecting a variation in the fluid within the one or more ducts to identifying the presence of a lesion within the one or more ducts.
